(19) United States
(12) Patent Application Publication
(10) Pub. No.: US 2009/0156661 A1
(43) Pub. Date: Jun. 18, 2009

(54) Compositions and Methods for Treatment and Prevention of Metabolic Syndrome and its Associated Conditions with Combinations of Flavonoids, Liminoids and Tocotrienols

(75) Inventor: Najla Guthrie, London (CA)

Correspondence Address: PATENT DOCKET ADMINISTRATOR;LOWENSTEIN SANDLER PC, 65 LIVINGSTON AVENUE, ROSELAND, NJ, 07068, US

(73) Assignee: KGK SYNERGIZE INC., London (CA)

(21) Appl. No.: 12/250,334

(22) Filed: Oct. 13, 2008

Related U.S. Application Data
(63) Continuation of application No. 11/595,558, filed on Nov. 10, 2006, now abandoned.
(60) Provisional application No. 60/735,660, filed on Nov. 10, 2005.

(51) Int. Cl. A61K 31/353; A61K 31/366

(52) U.S. Cl. 514/453; 514/456

(57) ABSTRACT

The present invention is directed to compositions and methods for the treatment and/or prevention of metabolic syndrome and its associated conditions, such as insulin resistance, which involve using a combination composition of limonoids, flavonoids and tocotrienols.

### RELATED APPLICATIONS

This application claims priority to U.S. Provisional Application No. 60/753,660, filed Nov. 10, 2005, the contents of which are hereby incorporated by reference in its entirety.

### BACKGROUND OF THE INVENTION

Metabolic syndrome, a condition thought to be caused by a combination of obesity, sedentary lifestyle, diet and genetics, has been found to increase the risk for cardiovascular disease and Type II diabetes. The main characteristics of this syndrome are abdominal obesity, atherogenic dyslipidemia (elevated blood triglycerides, reduced HDL cholesterol), elevated blood pressure, insulin resistance (IR) (with or without glucose intolerance), prothrombotic and proinflammatory states and endothelial dysfunction. During the past 20 years, metabolic syndrome has become highly prevalent in North America, currently affecting an estimated 50% of the population older than 60 years.

Insulin resistance, one of the characteristics of metabolic syndrome, is defined as an impaired ability of insulin to stimulate glucose uptake and lipolysis and to modulate liver and muscle lipid metabolism. In animals and humans, insulin resistance syndrome leads to compensatory hyperinsulinemia and to various defects in lipid metabolism such as enhanced secretion of atherogenic, triacylglycerol-rich very low-density lipoproteins (VLDL), increased liberation of nonesterified fatty acids (NEFA) from adipose tissue and increased accumulation of triacylglycerols in the liver.

Current therapies in prevention and treatment of Type II diabetes include diet and drugs. Dietary strategies designed to diminish the risk of heart disease associated with insulin resistance syndrome and Type II diabetes are currently not well established. The most common approach is the recommendation to lower intake of total calories, especially fat and sugar, and to increase intake of fibers. The typical pharmacologic approach to the treatment of this disease focuses on drugs targeting obesity, glucose-lowering medications (e.g., metformin and acarbose) and more recently, insulin sensitizers such as PPAR-α and PPAR-γ activators, fibrates and thiazolidienodiones (TZDs). Unfortunately, therapies involving existing drugs have limited efficacy or tolerability and show significant side effects. There exists a need to provide a safe and effective method of treating metabolic syndrome and the diseases associated with it.

### OBJECTS AND SUMMARY OF THE INVENTION

It is an object of the present invention to provide a composition including a combination of limonoid(s), flavonoid(s) and tocotrienol(s) suitable for the treatment of metabolic syndrome.

It is an object of certain embodiments of the present invention to provide a method of treating metabolic syndrome in humans by administering a composition including a combination of limonoid(s), flavonoid(s) and tocotrienol(s) to human patients suffering from such a condition.

The above mentioned objects and others are achieved by virtue of the present invention which is directed in part to compositions and methods for the treatment and/or prevention of metabolic syndrome and its associated conditions, such as insulin resistance which involve using a pharmaceutical composition comprising an effective amount of at least one compound selected from the group consisting of at least one limonoid, at least one flavonoid, at least one tocotrienol, and combination thereof.

In certain embodiments, the present invention is directed to a pharmaceutical composition comprising an effective amount of at least one compound selected from the group consisting of a limonoid, a flavonoid, a tocotrienol, and a combination thereof which after about 4 weeks of administration of said composition to humans, said composition provides at least about 10 percent increase in mean time to maximum plasma concentration (Tₘₐₓ) of plasma insulin in said humans after administration of an oral glucose tolerance test as compared to the mean time to maximum plasma concentration (Tₘₐₓ) of plasma insulin after an oral glucose tolerance test prior to said 4 week interval.

In certain embodiments, the present invention is directed to a pharmaceutical composition comprising a effective amount of a combination of compounds comprising at least one limonoid, at least one flavonoid and at least one tocotrienol, which after about 4 weeks of administration of said composition to humans, said composition provides at least about 10 percent increase in mean time to maximum plasma concentration (Tₘₐₓ) of plasma insulin in said humans after administration of an oral glucose tolerance test as compared to the mean time to maximum plasma concentration (Tₘₐₓ) of plasma insulin after an oral glucose tolerance test prior to said 4 week interval.

In certain embodiments, the present invention is directed to a pharmaceutical composition comprising an effective amount of a combination of compounds comprising at least one limonoid, at least one flavonoid and at least one tocotrienol, which after about 4 weeks of administration of said composition to humans, said composition provides from about 20 to about 70 percent increase in mean time to maximum plasma concentration (Tₘₐₓ) of plasma insulin in said humans after administration of an oral glucose tolerance test as compared to the mean time to maximum plasma concentration (Tₘₐₓ) of plasma insulin after an oral glucose tolerance test prior to said 4 week interval.

In certain embodiments, the present invention is directed to a pharmaceutical composition comprising an effective amount of at least one compound selected from the group consisting of a limonoid, a flavonoid, a tocotrienol, and a combination thereof which after about 4 weeks of administration of said composition to humans, said composition provides at least about a 5 percent decrease in mean maximum plasma concentration (Cₘₐₓ) of plasma insulin in said humans after administration of an oral glucose tolerance test as compared to the mean maximum plasma concentration (Cₘₐₓ) of plasma insulin after an oral glucose tolerance test prior to said 4 week interval.

In certain embodiments, the present invention is directed to a pharmaceutical composition comprising an effective amount of a combination of compounds comprising at least one limonoid, at least one flavonoid and at least one tocotrienol, which after about 4 weeks of administration of said composition to humans, said composition provides at least about a 5 percent decrease in mean maximum plasma concentration (Cₘₐₓ) of plasma insulin in said humans after administration of an oral glucose tolerance test as compared to the mean maximum plasma concentration (Cₘₐₓ) of plasma insulin after an oral glucose tolerance test prior to said 4 week interval.

In certain embodiments, the present invention is directed to a pharmaceutical composition comprising an effective amount of a combination of compounds comprising at least one limonoid, at least one flavonoid and at least one tocotrienol, which after about 4 weeks of administration of said composition to humans, said composition provides from about 5 to about 60 percent decrease in mean maximum plasma concentration (Cₘₐₓ) of plasma insulin in said humans after administration of an oral glucose tolerance test as compared to the mean maximum plasma concentration (Cₘₐₓ) of plasma insulin after an oral glucose tolerance test prior to said 4 week interval.

In certain embodiments, the present invention is directed to a pharmaceutical composition comprising an effective amount of at least one compound selected from the group consisting of a limonoid, a flavonoid, a tocotrienol, and a combination thereof which after about 4 weeks of administration of said composition to humans, said composition provides at least about a 5 percent decrease in mean AUC₀₋₂ₕ of plasma insulin in said humans after administration of an oral glucose tolerance test as compared to the mean AUC₀₋₂ₕ of plasma insulin after an oral glucose tolerance test prior to said 4 week interval.

In certain embodiments, the present invention is directed to a pharmaceutical composition comprising an effective amount of a combination of compounds comprising at least one limonoid, at least one flavonoid and at least one tocotrienol, which after about 4 weeks of administration of said composition to humans, said composition provides at least about a 5 percent decrease in mean AUC₀₋₂ₕ of plasma insulin in said humans after administration of an oral glucose tolerance test as compared to the mean AUC₀₋₂ₕ of plasma insulin after an oral glucose tolerance test prior to said 4 week interval.

In certain embodiments, the present invention is directed to a pharmaceutical composition comprising an effective amount of a combination of compounds comprising at least one limonoid, at least one flavonoid and at least one tocotrienol, which after about 4 weeks of administration of said composition to humans, said composition provides from about 5 to 55 percent decrease in mean AUC₀₋₂ₕ of plasma insulin in said human patient after administration of an oral glucose tolerance test as compared to the mean AUC₀₋₂ₕ of plasma insulin after in oral glucose tolerance test prior to said 4 week interval.

In certain preferred embodiment, the composition provides from about 30 to about 50, more preferably from about 40 to about 45 percent increase in time to mean maximum plasma concentration (Tₘₐₓ) of plasma insulin in humans after administration of an oral glucose tolerance test as compared to the time to mean maximum plasma concentration (Tₘₐₓ) of plasma insulin after an oral glucose tolerance test prior to said 4 week interval.

In certain preferred embodiments, the composition provides from about 10 to about 40, more preferably from about 15 to about 20 percent decrease in mean maximum plasma concentration (Cₘₐₓ) of plasma insulin in humans after administration of an oral glucose tolerance test as compared to the mean maximum plasma concentration (Cₘₐₓ) of plasma insulin after an oral glucose tolerance test prior to said 4 week interval.

In certain preferred embodiments, the composition provides about 5 to about 30 percent, more preferably about 8 to about 15 percent decrease in mean AUC₀₋₂ₕ of plasma insulin in humans after administration of an oral glucose tolerance test as compared to the mean AUC₀₋₂ₕ of plasma insulin after an oral glucose tolerance test prior to said 4 week interval.

In certain embodiments, the present invention is directed to a pharmaceutical composition comprising an effective amount of at least one compound selected from the group consisting of a limonoid, a flavonoid, a tocotrienol, and a combination thereof which after about 4 weeks of administration of said composition to humans, said composition provides at least about 10 percent increase in mean time to maximum plasma concentration (Tₘₐₓ) of plasma glucose in said humans alter administration of an oral glucose tolerance test as compared to the mean time to maximum plasma concentration (Tₘₐₓ) of plasma glucose after an oral glucose tolerance test prior to said 4 week interval.

In certain embodiments, the present invention is directed to a pharmaceutical composition comprising a effective amount of a combination of compounds comprising at least one limonoid, at least one flavonoid and at least one tocotrienol, which after about 4 weeks of administration of said composition to humans, said composition provides at least about 10 percent increase in mean time to maximum plasma concentration (Tₘₐₓ) of plasma glucose in said humans after administration of an oral glucose tolerance test as compared to the mean time to maximum plasma concentration (Tₘₐₓ) of plasma glucose after an oral glucose tolerance test prior to said 4 week interval.

In certain embodiments, the present invention is directed to a pharmaceutical composition comprising an effective amount of a combination of compounds comprising at least one limonoid, at least one flavonoid and at least one tocotrienol, which after about 4 weeks of administration of said composition to humans, said composition provides from about 20 to about 70 percent increase in mean time to maximum plasma concentration (Tₘₐₓ) of plasma glucose in said humans after administration of an oral glucose tolerance test as compared to the mean time to maximum plasma concentration (Tₘₐₓ) of plasma glucose after an oral glucose tolerance test prior to said 4 week interval.

In certain preferred embodiment, the composition provides from about 30 to about 50, more preferably from about 40 to about 45 percent increase in time to mean maximum plasma concentration (Tₘₐₓ) of plasma glucose in humans after administration of an oral glucose tolerance test as compared to the time to mean maximum plasma concentration (Tₘₐₓ) of plasma glucose after an oral glucose tolerance test prior to said 4 week interval.

In certain embodiments, the combination of at least one limonoid, at least one flavonoid and at least one tocotrienol is a synergistic combination.

In certain preferred embodiments, the flavonoid is selected from the group consisting of polymethoxyflavones, naringin and hesperidin.

In certain embodiments, the polymethoxyflavones are a combination of nobiletin, HMF and tangeretin. In certain preferred embodiments, the nobiletin, HMF and tangeretin are present in a ratio of about 7-9:1-:3:0.3-1.5. In more preferred embodiments, the nobiletin, HMF and tangeretin are present in a ratio of about 3:2:1.

In certain embodiments, the composition comprises a combination of nobiletin, HMF and tangeretin and at least one tocotrienol.

In certain embodiments, the composition comprises a combination of nobiletin, HMF and tangeretin and diabetinol.

In certain embodiments, the composition is suitable for administration intravenously, intraperitoneally, subcutaneously, intramuscularly, intrathecally, orally, rectally, topically or by inhalation.

In certain embodiments, the composition is in the form of a tablet, a capsule, a solution, a suspension, or an emulsion.

In certain embodiments, the composition further comprises soy protein.

In certain embodiments, the composition comprises from about 200 to about 5000 mg of at least one flavonoid; from about 1 to about 500 mg of at least one limonoid; from about 1 to about 1200 mg of at least one tocotrienol; and optionally from about 1 to about 500 g of soy protein.

In certain embodiments, the composition provides a decrease in serum insulin levels of at least 5%, at least 10%, at leas 20%, or at least 30% after 4 weeks of treatment as compared to a fructose 60% control group. In certain embodiments, the composition provides a decrease in serum from about 5% to about 40% after 4 weeks of treatment as compared to a fructose 60% control group. In certain embodiments, the composition provides a decrease in serum from about 10% to about 30% after 4 weeks of treatment as compared to a fructose 60% control group. In certain embodiments, the composition provides a decrease in serum insulin levels of from about 12% to about 28% after 4 weeks of treatment as compared to a fructose 60% control group.

In certain embodiments, the composition provides a decrease in serum triglyceride levels of at least 5%, at least 101%, at least 20%, at least 30%, at least 40%, or at least 50% after 4 weeks of treatment as compared to a fructose 60% control group. In certain embodiments, the composition provides a decrease in serum triglyceride levels of from about 10% to about 65% after 4 weeks of treatment as compared to a fructose 60% control group. In certain embodiments, the composition provides a decrease in serum triglyceride levels of from about 20% to about 55% after 4 week, of treatment as compared to a fructose 60% control group. In certain embodiments, the composition provides a decrease in serum triglyceride levels of from about 24% to about 48% after 4 weeks of treatment as compared to a fructose 60% control group.

In certain embodiments, the composition provides a decrease in serum cholesterol levels of at least 2%, at least 10%, or at least 20% after 4 weeks of treatment as compared to a fructose 60%/o control group. In certain embodiments, the composition provides a decrease in serum cholesterol levels of from about 2% to about 30% after 4 weeks of treatment as compared to a fructose 60% control group. In certain embodiments, the composition provides a decrease in serum cholesterol levels of from about 5% to about 20% after 4 weeks of treatment as compared to a fructose 60% control group. In certain embodiments, the composition provides a decrease in serum cholesterol levels of about 10% after 4 weeks of treatment as compared to a fructose 60% control group.

In certain embodiments, the composition provides a decrease in serum glucose levels of at least 5%, at least 10%, or at least 20% after 4 weeks of treatment as compared to a fructose 60% control group. In certain embodiments, the composition provides a decrease in serum glucose levels of from about 10% to about 40% after 4 weeks of treatment as compared to a fructose 60% control group. In certain embodiments, the composition provides a decrease in serum glucose levels of from about 20% to about 30% after 4 weeks of treatment as compared to a fructose 60% control group. In certain embodiments, the composition provides a decrease in serum glucose levels of about 25% after 4 weeks of treatment as compared to a fructose 60% control group.

In certain embodiments, the present invention is further directed to a method for treating a human patient with insulin resistance syndrome comprising administering to a human patient any of the compositions described herein.

In certain embodiments, the present invention is further directed to a method for treating a human patient with metabolic syndrome comprising administering to a human patient any of the compositions described herein.

The term “pharmaceutical composition” means a composition comprising a compound of the invention in combination with at least one additional pharmaceutical carrier, i.e., adjuvant, excipient or vehicle, such as diluents, preserving agents, fillers, flow regulating agents, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Ingredients listed in Remington's Pharmaceutical Sciences, 18^{th} ed., Mack Publishing Company, Easton, Pa. (1990) for example, may be used.

### BRIEF DESCRIPTION OF THE DRAWINGS

Glucose Tolerance Test (OGTT) between treatment group and placebo group before and after a 4-wk supplementation period.

FIG. 2 depicts a comparison of mean (±SD) plasma insulin responses after Oral Glucose Tolerance Test (OGTT) between treatment group and placebo group before and after a 4-wk supplementation period.

FIG. 3 depicts correlations between insulin pharmacokinetic parameters and BMI. (A) Change in insulin AUC₀₋₂ₕ vs. BMI, (B) Change in insulin Cₘₐₓ vs. BMI, (C) Change in insulin Tₘₐₓ vs. BMI.

FIG. 4 depicts a graph of the insulin levels from Example 4.

FIG. 5 depicts a graph of the triglyceride levels from Example 4.

FIG. 6 depicts a graph of the cholesterol levels from Example 4.

FIG. 7 depicts a graph of the glucose levels from Example 4.

### DETAILED DESCRIPTION OF THE PREFERRED EMBODIMENTS

The present invention relates to compositions and methods for the treatment of metabolic syndrome and its accompanying characteristics, including insulin resistance, with combinations of certain flavonoids, limonoids, tocotrienols and/or soy protein. Flavonoids are polyphenolic compounds that occur ubiquitously in plant foods especially in orange, grapefruit and tangerine. Limonoids are a group of chemically related triterpene derivatives found in the Rutaceae and Meliaceae families. Limonoids are among the bitter principals in citrus juices such as lemon, lime, orange and grapefruit. Tocotrienols are present in palm oil and are a form of vitamin E having an unsaturated side chain.

### Flavonoids

Flavonoids are polyphenolic compounds that are found in plant foods, especially in oranges, grapefruits and tangerines. Polymethoxyflavones (PMFs) are flavonoid compounds having multiple methoxy substituents. Various beneficial effects of flavonoids are described in U.S. Pat. Nos. 6,251,400 and 6,239,114 and in PCT Publication Number WO 01/70029, the disclosures of which are hereby incorporated by reference in their entireties. Other beneficial effects of flavonoid derivatives are discussed in U.S. Pat. Nos. 4,591,600; 5,855,892; and 6,096,364, the disclosures of which are also hereby incorporated by reference in their entireties.

The flavonoids present in citrus juices such as orange and grapefruit include, but are not limited to, hesperetin and naringenin respectively.

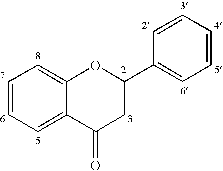

| | 5 | 7 | 3′ | 4′ |
|---|---|---|---|---|
| HESPERETIN | OH | OH | OH | OCH₃ |
| NARINGENIN | OH | OH | — | O |

### Limonoids

Limonoids are a group of chemically related triterpene derivatives found in the Rutaceae and Meliaceae families. Limonoids are among the bitter principles found in citrus fruits such as lemons, lime, orange and grapefruit. They are also present as glucose derivatives in mature fruit tissues and seed, and are one of the major secondary metabolites present in citrus.

Citrus fruit tissues and byproducts of juice processing such as peels and molasses are sources of limonoid glucosides and citrus seed contain high concentrations of both limonoid aglycones and glucosides. Limonoid aglycones in the fruit tissues gradually disappear during the late stages of fruit growth and maturation.

Thirty-eight limonoid aglycones have been isolated from citrus. The limonoids are present in three different forms: the dilactone (I) is present as the open D-ring form (monolactone), the limonoate A-ring lactone (II) and the glucoside form (III). Only the monolactones and glucosides are present in fruit tissues. (Hasegawa S. et al., 1994, in Food Phytochemicals for Cancer Prevention I, eds M-T. Huang et al. American Chemical Society, 198-207).

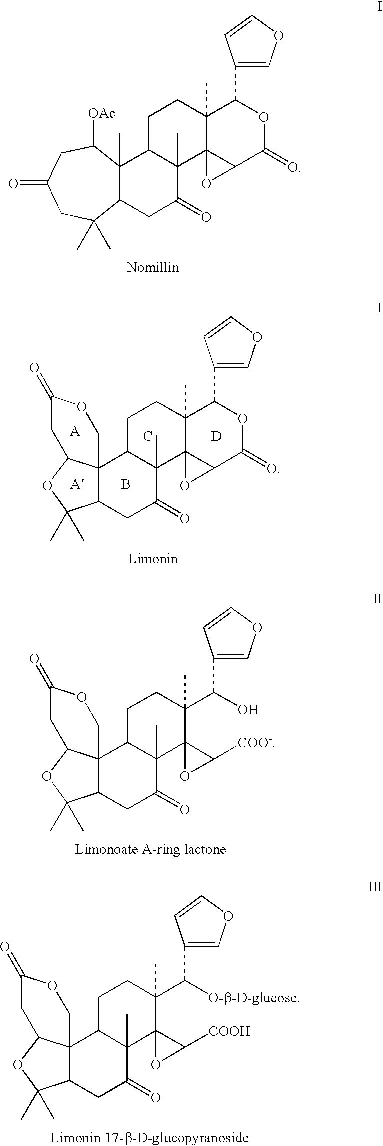

Compound III is the predominant limonoid glucoside found in all juice samples. In orange juice it comprises 56% of the total limonoid glucosides present, while in grapefruit and lemon juices, it comprises an average of 63% to 66% respectively. Procedures for the extraction and isolation of both aglycones and glucosides have been established to obtain concentrated sources of various limonoids (Lam, L. K. T. et al., 1994, in Food Phytochemicals for Cancer Prevention, eds. M. Huang, T. Osawa, C. Ho and R. T. Rosen, ACS Symposium Series 546, p 209).

Tocotrienols are present in palm oil and are a form of vitamin E having an unsaturated side chain. They include, but are not limited to alpha-tocotrienol, gamma-tocotrienol or delta-tocotrienol.

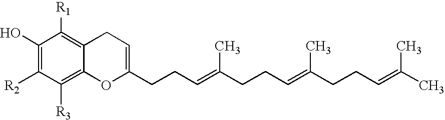

| | R1 | R2 | R3 |
|---|---|---|---|
| α-tocotrienol | CH₃ | CH₃ | CH₃ |
| γ-tocotrienol | H | CH₃ | CH₃ |
| δ-tocotrienol | H | H | CH₃ |

### Soy Protein

Soy protein is a complete protein derived from soy beans. Soybean isoflavones for example, genistein, which is a minor component of soy protein preparations may have cholesterol-lowering effects (Kurowska, E. M. et al., 1990, J. Nutr. 120:831-836). Recent studies suggest that soy protein and soy isoflavones, genistein and daidzein, might also be beneficial in insulin resistance and Type II diabetes.

Citrus limonoids, citrus flavonoids, tocotrienols or soy proteins may be formulated into pharmaceutical preparations for administration to mammals for prevention and treatment of insulin resistance, cardiovascular disease, hypercholesterolemia or atherosclerosis.

Many of the citrus limonoids, flavonoids, tocotrienols or soy proteins may be provided as compounds with pharmaceutically compatible counterions, a form in which they may be soluble.

Formulations containing the citrus limonoids, citrus flavonoids, tocotrienols and/or soy proteins of the present invention may by administered by any acceptable means including orally, transdermally, rectally, intravenously, intramuscularly, intraperitoneally, subcutaneously, topically, by inhalation or any other means. The oral administration means is preferred. Formulations suitable for oral administration are commonly known and include liquid solutions of the active compounds dissolved in a diluent such as, for example, saline, water, PEG 400, etc. Solid forms of the compounds for oral administration include capsules or tablets, each comprising the active ingredients and commonly known adjuvants. The active ingredients in the solid dosage form may be present in the form of solids, granules, gelatins, suspensions, and/or emulsions, as will be apparent to persons skilled in the art. The pharmaceutical compositions also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Formulations suitable for parenteral administration include aqueous and non aqueous isotonic sterile solutions containing buffers, antioxidants, preservatives and any other known adjuvants.

Useful solutions for oral or parenteral administration can be prepared by any of the methods well known in the pharmaceutical art, described, for example, in Remington's Pharmaceutical Sciences, 18th ed. (Mack Publishing Company, 1990). Formulations for parenteral administration can also include glycocholate for buccal administration, methoxysalicylate for rectal administration, or citric acid for vaginal administration. The parenteral preparation can be enclosed in ampoules, disposable syringes or multiple dose vials made of glass or plastic. Suppositories for rectal administration also can be prepared by mixing the drug with a non-irritating excipient such as cocoa butter, other glycerides, or other compositions which are solid at room temperature and liquid at body temperatures. Formulations also can include, for example, polyalkylene glycols such as polyethylene glycol, oils of vegetable origin, and hydrogenated naphthalenes. Formulations for direct administration can include glycerol and other compositions of high viscosity. Other potentially useful parenteral carriers for these drugs include ethylene-vinyl acetate copolymer particles, osmotic pumps, implantable infusion systems, and liposomes. Formulations for inhalation administration can contain as excipients, for example, lactose, or can be aqueous solutions containing, for example, polyoxyethylene-9-lauryl ether, glycocholate and deoxycholate, or oily solutions for administration in the form of nasal drops, or as a gel to be applied intranasally. Retention enemas also can be used for rectal delivery.

Formulations of the present invention suitable for oral administration can be in the form of: discrete units such as capsules, gelatin capsules, sachets, tablets, troches, or lozenges, each containing a predetermined amount of the drug; a powder or granular composition; a solution or a suspension in an aqueous liquid or non-aqueous liquid; or an oil-in-water emulsion or a water-in-oil emulsion. The drug can also be administered in the form of a bolus, electuary or paste. A tablet can be made by compressing or molding the drug optionally with one or more accessory ingredients. Compressed tablets can be prepared by compressing, in a suitable machine, the drug in a free-flowing form such as a powder or granules, optionally mixed by a binder, lubricant, inert diluent, surface active or dispersing agent. Molded tablets may be made by molding, in a suitable machine, a mixture of the powdered drug and suitable carrier moistened with an inert liquid diluent.

Oral compositions generally include an inert diluent or an edible carrier. For the purpose of oral therapeutic administration, the active compound can be incorporated with excipients. Oral compositions prepared using a fluid carrier for use as a mouthwash include the compound in the fluid carrier and are applied orally and swished and expectorated or swallowed. Pharmaceutically compatible birding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of the drug that can be in microcrystalline form, for example, in the form of an aqueous microcrystalline suspension. Liposomal formulations or biodegradable polymer systems can also be used to present the drug for both intra-articular and ophthalmic administration.

Formulations suitable for topical administration, including eye treatment, include liquid or semi-liquid preparations such as liniments, lotions, gels, applicants, oil-in-water or water-in-oil emulsions such as creams, ointments or pastes; or solutions or suspensions such as drops. Formulations for topical administration to the skin surface can be prepared by dispersing the drug with a dermatologically acceptable carrier such as a lotion, cream, ointment or soap. Particularly useful are carriers capable of forming a film or layer over the skin to localize application and inhibit removal. For topical administration to internal tissue surfaces, the agent can be dispersed in a liquid tissue adhesive or other substance known to enhance adsorption to a tissue surface. For example, hydroxypropylcellulose or fibrinogen/thrombin solutions can be used to advantage. Alternatively, tissue-coating solutions, such as pectin-containing formulations can be used.

For inhalation treatments, inhalation of powder (self-propelling or spray formulations) dispensed with a spray can, a nebulizer, or an atomizer can be used. Such formulations can be in the form of a fine powder for pulmonary administration from a powder inhalation device or self-propelling powder-dispensing formulations. In the case of self-propelling solution and spray formulations, the effect can be achieved either by choice of a valve having the desired spray characteristics (i.e., being capable of producing a spray having the desired particle size) or by incorporating the active ingredient as a suspended powder in controlled particle size. For administration by inhalation, the compounds also can be delivered in the form of an aerosol spray from pressured container or dispenser which contains a suitable propellant, e.g., a gas such as carbon dioxide, or a nebulizer.

Systemic administration also can be by transmucosal or transdermal means. For transmucosal or transdermal administration, penetrants appropriate to the barrier to be permeated are used in the formulation. Such penetrants generally are known in the art, and include, for example, for transmucosal administration, detergents and bile salts. Transmucosal administration can be accomplished through the use of nasal sprays or suppositories. For transdermal administration, the active compounds typically are formulated into ointments, salves, gels, or creams as generally known in the art.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

Oral or parenteral compositions can be formulated in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. Furthermore, administration can be by periodic injections of a bolus, or can be made more continuous by intravenous, intramuscular or intraperitoneal administration from an external reservoir (e.g., an intravenous bag).

Where adhesion to a tissue surface is desired the composition can include the drug dispersed in a fibrinogen-thrombin composition or other bioadhesive. The compound then can be painted, sprayed or otherwise applied to the desired tissue surface. Alternatively, the drugs can be formulated for parenteral or oral administration to humans or other mammals, for example, in effective amounts, e.g., amounts that provide appropriate concentrations of the drug to target tissue for a time sufficient to induce the desired effect.

Patient dosages for oral administration of citrus limonoids range from 1-500 mg/day, commonly 1-100 mg/day, and typically from 1-100 mg/day. Stated in terms of patient body weight, usual dosages range from 0.01-10 mg/kg/day, commonly from 0.01-2.0 mg/kg/day, typically from 0.01 to 2.0 mg/kg/day.

Patient dosages for oral administration of citrus flavonoids range from 200-5000 mg/day, commonly 1000-2000 mg/day, and typically from 500-1500 mg/day. Stated in terms of patient body weight, usual dosages range from 15-70 mg/kg/day, commonly from 15-30 mg/kg/day, typically from 7-21 mg/kg/day.

Patient dosages for oral administration of tocotrienols range from 1-1200 mg/day, commonly 1-100 mg/day, and typically from 1-60 mg/day. Stated in terms of patient body weight, usual dosages range from 0.01-20 mg/kg/day, commonly from 0.01-2.0 mg/kg/day, typically from 0.01 to 1/0 mg/kg/day.

Patient dosages for oral administration of soy protein range from 1-500 g/day, commonly 25-250 g/day, and typically from 25-100 g/day.

In certain preferred embodiments, the composition comprises about 300 mg polymethoxyflavones, about 100 mg hesperidin, about 100 mg naringin, about 30 mg limonoids and about 10 mg tocotrienols.

The above disclosure generally describes the present invention. A more complete understanding can be obtained by reference to the following specific examples which are provided herein for purposes of illustration only, and are not intended to limit the scope of the invention.

### EXAMPLES

It is noted that interspecies studies involving pharmacokinetic parameters may be used to predict certain outcomes in untested species, as described in detail in Mordenti, J. “Man versus Beast: Pharmacokinetic Scaling in Mammals”, J of Pharm. Science, Vol. 75, No. 11, November 1986, pp. 1028-1040, the disclosure of which is hereby incorporated by reference in its entirety.

### Example 1

Metabolic responses to three dietary supplements, alone and in combinations with PMFs, in the hamster model of fructose-induced IR were evaluated. The compounds investigated were: i) a mixture of hesperidin naringin; ii) tocotrienols, natural analogues of vitamin E abundant in palm oil and in cereal grains, administered as tocotrienol-rich fraction (TRF) from palm oil; and, iii) soy protein containing natural levels of isoflavones. In 60% of the fructose-fed hamsters, a supplementation with a 3% mixture of hesperidin and naringin (1:1, w/w) tended to reduce fasting plasma glucose and to improve responses to i.p. glucose challenge without affecting blood lipids, whereas a supplementation with 1% TRF had little effect on glucose but reduced blood cholesterol. Feeding both supplements together induced more pronounced lowering of fasting glucose concentrations and moderately improved glucose tolerance. Beneficial changes in glucose metabolism after treatment with combinations of supplements were further enhanced in hamsters co-administered 1% PMF and 1% TRF or in animals fed citrus flavonoids alone or citrus flavonoids with tocotrienols, on the background of soy protein instead of casein diet. Thus, a combination of 1% PMF and 1% TRF added to the casein-based diet substantially reduced fasting glucose concentrations and improved glucose tolerance (while also reducing blood lipids and NEFA). Even more pronounced decreases in fasting glucose and in glucose tolerance were observed in animals fed PMF, TRF and/or hesperidin/naringin (as single supplements or as pairs, a flavonoid plus TRF) in combination with soy protein (unpublished). The above results suggest that not only PMF but also hesperidin and naringin may contribute to improvement in dyslipidemia and glycemic control when co-administered with TRF and/or with soy protein.

### Example 2

The blood lipid responses to dietary supplementation with PMF and TRF, with or without addition of citrus limonoids (bitter compounds abundant in citrus seeds) were evaluated in subjects with moderate hypercholesterolemia. Results showed that a 4-week supplementation with capsules containing 200 mg PMF, 300 mg tocotrienols and 200 mg mixed limonoids (mainly limonin, nomilin and their glycosides) tended to improve blood lipid profiles by lowering plasma TG and increasing HDL cholesterol. Although plasma glucose and insulin levels were not measured, the beneficial lipid-lowering responses appeared to be more pronounced in individuals with baseline characteristics consistent with metabolic syndrome.

### Example 3

A daily dose of the phytochemical supplement was prepared containing the following amounts of individual components:

| | |
|---|---|
| Polymethoxyflavones (PMF) | 300 mg |
| Hesperidin (Hesp) | 100 mg |
| Naringin (Nar) | 100 mg |
| Limonoids (Lim) | 30 mg |
| Tocotrienols (T3) | 10 mg |

The study was a double-blind, placebo-controlled, and randomized parallel group design. Ten healthy male and female adults (26-59), three men and seven women, were included. Prior to the start of the study, subjects had a physical exam, which included anthropometric and blood pressure measurements and routine blood tests (lipid profile and fasting glucose). Women of childbearing age also had a urine pregnancy test. Subjects with metabolic syndrome were advised to avoid taking dietary supplements 4 weeks prior to treatment period and to maintain normal dietary and exercise habits over the course of the study. They were also asked to avoid caffeine-containing products and strenuous exercise 72 h prior to test days.

During the baseline visit (day 0) participants were asked to conduct OGTT, which included providing a fasting blood sample, consuming a standard 25 g glucose beverage and providing additional blood samples at 15, 30, 45, 60, 90 and 120 min for determination of plasma glucose and insulin concentrations. After completion of the test, subjects were randomly divided into 2 groups which received either test product or placebo capsules for the following 4 wk period. At the end of the 4-week treatment period, the same OGTT was conducted, except, the last daily dose of capsules was administered together with a standard 25 g glucose drink. During the 4-wk visit, subjects returned any unused capsules and side effect diaries.

### Results

Baseline characteristic of the study subjects (6 on active treatment and 4 on placebo) are presented in Table 1. The initial body mass index was significantly higher in the treatment group than in the control group while the initial blood TG concentration was significantly higher in the control group than in the treatment group. Other baseline parameters were not statistically different between the groups.

TABLE 1
Baseline characteristics of subjects in OGTT study (Mean ± SD)

| Parameter | Treatment (n = 6) | Placebo (n = 4) |
|---|---|---|
| Females | 5 | 2 |
| Males | 1 | 2 |
| Age (y) | 42.6 ± 14.7 | 48.0 ± 8.5 |
| Body Mass Index (kg/m²) | 36.3 ± 2.4 | 31.0 ± 1.4** |
| Waist circumference (cm) | 110.7 ± 11.7 | 102.0 ± 4.5 |
| Systolic blood pressure (mmHg) | 137 ± 3 | 136 ± 5 |
| Diastolic blood pressure (mmHg) | 81 ± 9 | 83 ± 7 |
| Total cholesterol (mmol/L) | 5.53 ± 1.17 | 6.50 ± 1.55 |
| HDL cholesterol (mmol/L) | 0.95 ± 0.22 | 0.82 ± 0.15 |
| LDL cholesterol (mmol/L) | 3.85 ± 1.06 | 3.70 ± 0.85 |
| Triglycerides (mmol/L) | 1.60 ± 0.37 | 3.26 ± 1.63* |
| Fasting glucose (mmol/L) | 6.02 ± 1.39 | 4.58 ± 0.15 |

*p < 0.05 by unpaired t-test.
**p < 0.01 by unpaired t-test.

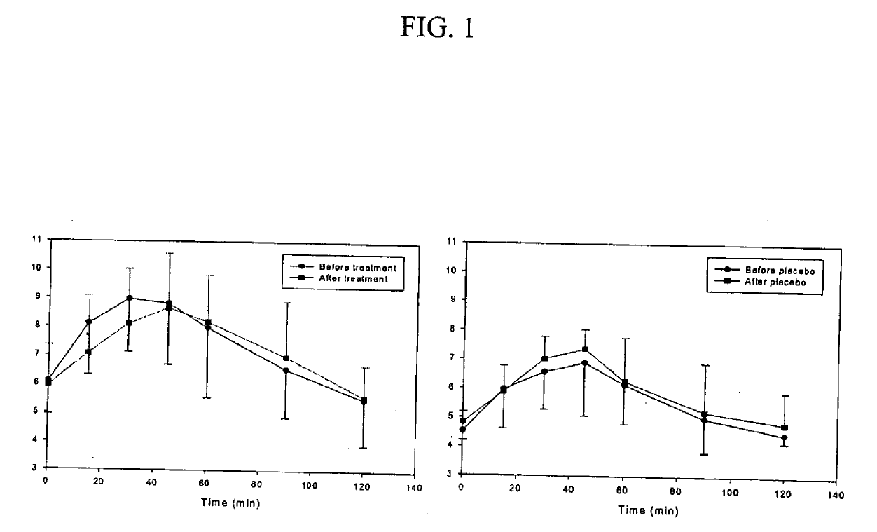

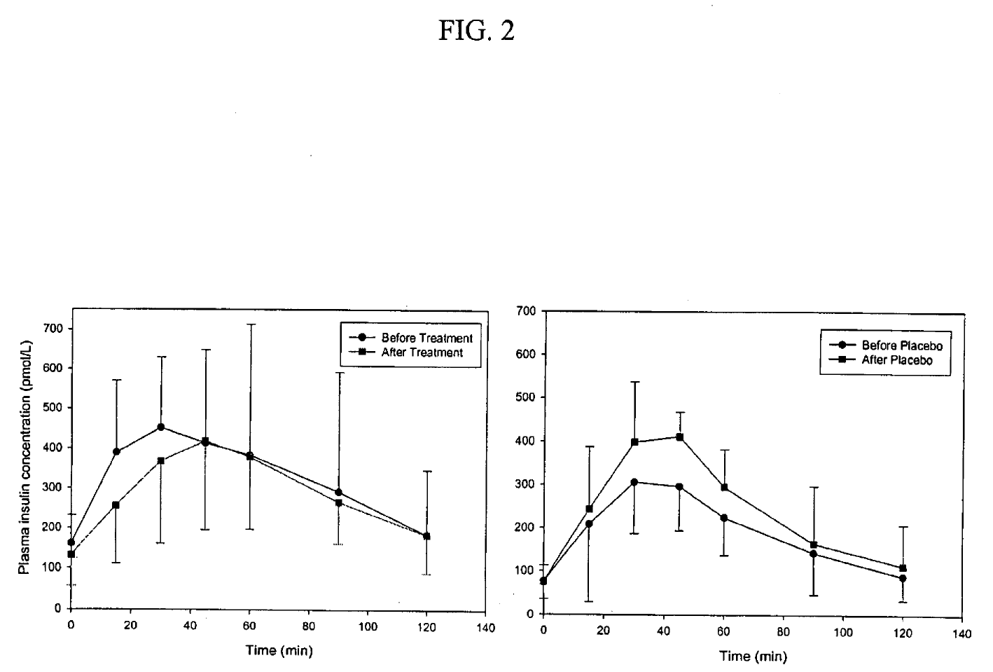

Plasma glucose and insulin responses observed during the initial vs. final OGTT in treatment vs. placebo group are presented in Tables 2-3. Mean plasma glucose and insulin OGTT profiles in both groups, before and after the 4-week supplementation period, are shown in FIGS. 1-2. Plasma glucose profiles tended to be improved by the treatment supplement but the time to maximum glucose concentration by approximately 14 min (43% increase in Tₘₐₓ) without affecting Cₘₐₓ and AUC₀₋₂ₕ. Treatment-induced changes in plasma insulin profiles showed similar but more pronounced beneficial trends. In this case, a 4-week supplementation was associated with a 22 min delay of insulin peak (53% increase in Tₘₐₓ) and also with moderate reductions in Cₘₐₓ and AUC₀₋₂ₕ (by 17% and 11%, respectively). In contrast, no improvement in OGTT insulin profiles was observed in the placebo group. Changes in insulin responses were not statistically significant.

TABLE 2
Plasma glucose pharmacokinetics after OGTT. A comparison between treatment
group and placebo group (Means ± SD).

| | Treatment | Treatment | Placebo | Placebo |
|---|---|---|---|---|
| Parameter | baseline | 4 weeks | baseline | 4 weeks |
| AUC_{0-2 h} (mmol × | 893.0 ± 209.9 | 880.0 ± 190.2 | 681.2 ± 111.1 | 711.0 ± 115.9 |
| min/L) | | | | |
| Cₘₐₓ (mmol/L) | 9.2 ± 1.7 | 8.9 ± 1.9 | 7.5 ± 1.4 | 7.6 ± 0.9 |
| Tₘₐₓ (min) | 32.5 ± 11.3 | 46.3 ± 12.0 | 41.3 ± 18.9 | 45.0 ± 12.2 |
| Tₘₐₓ change (%) | | +43% | | |

TABLE 3
Plasma insulin pharmacokinetics after OGTT. A comparison between treatment group
and placebo group (Means ± SD).

| | Treatment | Treatment | | Placebo |
|---|---|---|---|---|
| Parameter | baseline | 4 weeks | Placebo baseline | 4 weeks |
| AUC_{0-2 h} (pmol × | 40,283 ± 25,571 | 36,008 ± 14,893 | 23,494 ± 10,025 | 29,709 ± 8,050 |
| min/L) | | | | |
| AUC_{0-2 h} change (%) | | −11% | | + |
| Cₘₐₓ (pmol/L) | 531.2 ± 285.8 | 439.0 ± 216.3 | 302.5 ± 96.3 | 452.5 ± 89.8 |
| Cₘₐₓ change (%) | | −17% | | + |
| Tₘₐₓ (min) | 37.5 ± 18.4 | 57.5 ± 17.5 | 48.8 ± 7.5 | 41.3 ± 14.4 |
| Tₘₐₓ change (%) | | +43% | | |

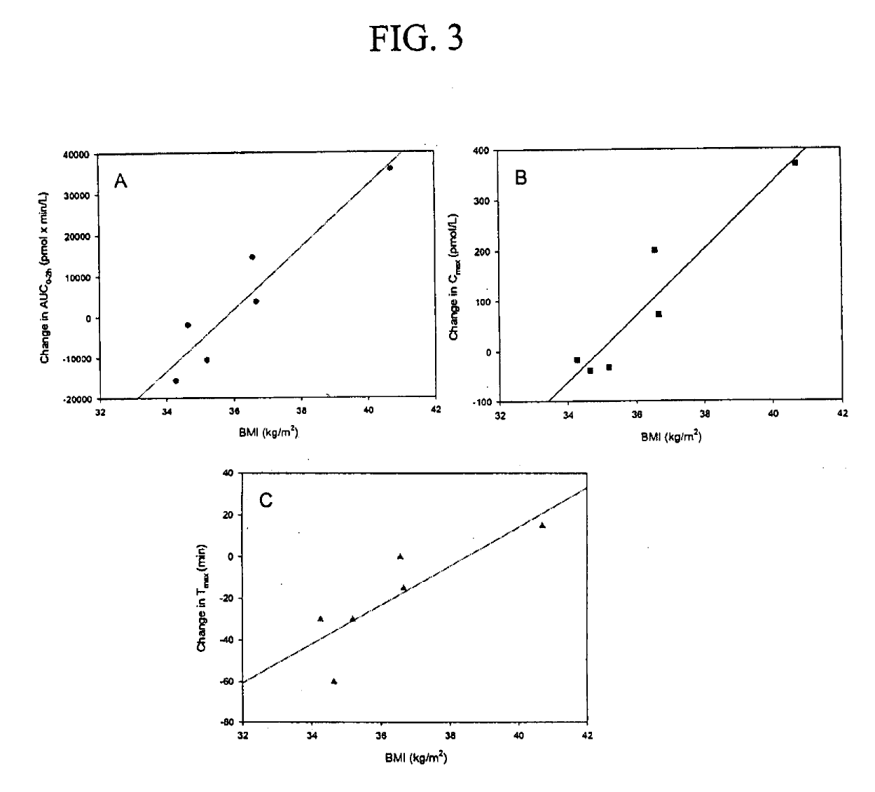

The correlations between changes in OGTT glucose and insulin responses (from before to after active treatment) and subjects' body mass index (BMI) are summarized in Table 4. Correlations obtained for insulin are also depicted in FIG. 3. For OGTT glucose profiles, only delays in Tₘₐₓ, but not changes in AUC₀₋₂ₕ or Cₘₐₓ associated with treatment were significantly correlated with subjects' BMI. In contrast, for OGTT insulin profiles, changes in all three parameters were significantly correlated with subjects' BMI. No adverse effects were observed in these subjects.

TABLE 4
Correlations between changes in glucose and insulin pharmacokinetic
parameters (from baseline to wk 4) and BMI.

| | r² | p value |
|---|---|---|
| Change in glucose AUC_{0-2 h} (pmol × | 0.5844 | 0.0767 |
| min/L) | | |
| Change in glucose Cₘₐₓ (pmol/L) | 0.5874 | 0.0754 |
| Change in glucose Tₘₐₓ (min) | 0.8660 | 0.0070** |
| Change in insulin AUC_{0-2 h} (pmol × min/L) | 0.8939 | 0.0440* |
| Change in insulin Cₘₐₓ (pmol/L) | 0.8949 | 0.0430* |
| Change in insulin Tₘₐₓ (min) | 0.7020 | 0.0372* |

### Example 4

The following compounds were used for this study:

1. Diabetinol (KGK Synergize, Lot # DIA240205)

2. PMF*-62% (batch #060104)

3. Tocotrienols (Tocomax 20% Carotech Batch # Tocomax_{—}1_{—}241 005)
The PMFs used in this study comprised approximately 8:1:2 of Nobiletin:

HMF:Tangeretin

### Experimental Design and Methods:

Induction of hypertriglyceridemia and insulin resistance:

Male Syrian Golden hamsters (Mesocricetus auratus) were fed a normal chow diet for 7 days to allow acclimatization to the new environment and recovery from the stress of shipping. Blood was collected at baseline; hamsters were weighed and randomized into 6 groups of 6 animals each:

A. CHOW—hamsters in this group were fed chow control for 6 weeks.

B. FRUC—hamsters in this group were fed fructose control (60%) diet DYETS #161506 for 6 weeks.

C. PMF—hamsters in this group were fed control (60%) diet DYETS #161506 for 2 weeks, then fructose (60%) DYETS # 161513+PMF (1%) for 4 weeks.

D. T-PMF—hamsters in this group were fed control (60%) diet DYETS #161506 for 2 weeks, then fructose (60%) DYETS # 161513+PMF+Tocotrienols (9:1; 1%) for 4 weeks.

E. DIA—hamsters in this group were fed control (60%) diet DYETS #161506 for 2 weeks, then fructose (60%) DYETS # 161513+Diabetinol (1%) for 4 weeks.

F. DIA-PMF—hamsters in this group were fed control (60%) diet DYETS #161506 for 2 weeks, then fructose (60%) DYET # 161513+PMF+Diabetinol (1:1; 1%) for 4 weeks.

### Fructose Diet (60%):

The 60% fructose diet, made by Dyets Inc. (diet # 161506), is the diet that has historically been used in the labs to induce insulin resistance in the hamsters. The components and their amounts per kg are: casein (220g), fructose (600g), corn oil (60g), cellulose fiber (70.9 g), L-arginine (1.0 g), L-tryptophan (1.1 g) and salt/vitamin mixes (45g). It also contains choline bitartrate (2g). There is no cholesterol.

Groups B-F were fed the DYET #161506 (powder) for 2 weeks:

| Ingredient | 60% Fructose diet (kg) | |
|---|---|---|
| Fructose (60%) | 600 9 | |
| Casein (22%) | 220 9 | |
| Corn oil (6%) | 60 9 | |
| Cellulose fiber (7.09%) | 70.9 9 | |
| L-arginine (0.1%) | 1.0 9 | |
| L-tryptophan (0.11%) | 1.1 9 | |
| Salt Mix # 260001 (3.5%) | 35 9 | |
| Vitamin Mix # 360001 (1.0%) | 10 | g |
| Choline bitartrate (0.2%) | 2 | g |

Groups C-F were fed the TEST diets with DYET #161513:

| Ingredient | 60% Fructose diet (kg) | |
|---|---|---|
| Fructose (60%) | 600 | g |
| Casein (22%) | 220 | g |
| Corn oil (6%) | 60 | g |
| Cellulose fiber (7.09%) | 60.9 | g |
| L-arginine (0.1%) | 1.0 | g |
| L-tryptophan (0.11%) | 1.1 | g |
| Salt Mix # 260001 (3.5%) | 35 | g |
| Vitamin Mix # 360001 (1.0%) | 10 | g |
| Choline bitartrate (0.2%) | 2 | g |
| TEST | 10 | g |

Calculation for TEST diets:

| Groups | PMF | Tocotrienols | Diabetinol |
|---|---|---|---|
| C - PMF | 10 g | | |
| D - T-PMF | 9 g | 1 g | |
| E - DIA | | | 10 g |
| F - DIA-PMF | 5 g | | 5 g |

One group of hamsters were fed the control diet (normal chow) and the rest of the groups were fed fructose-enriched diet (hamster diet with 60% fructose, Dyets Inc., no. 161506, Bethlehem, Pa.) for two weeks to induce hypertriglyceridemia and insulin resistance as previously described. During these two weeks hamster weight was monitored every 2 days.

The hamsters were assessed at the end of two-weeks of fructose feeding for the development of their insulin resistance status by monitoring body weight, glucose, triglyceride, cholesterol and insulin. Glucose was determined on whole blood using a glucometer.

### Sample and Data Collection and Analyses:

Blood samples from 16 hour fasted hamsters were collected from the retroorbital venous plexus into EDT A-coated and non-anticoagulant tubes at baseline (first day of fructose feeding), after week 2 before starting on experimental diets. At the end of the study, the blood samples were collected by cardiac puncture. Serum was collected after centrifugation at 3,000×g for 20 min at 4 DC and kept at −80 DC until use for the determination of biochemical markers. Glucose was determined on whole blood using a glucometer.

Routine cage-side observations were made on all animals throughout the study for general signs of pharmacologic effects, morbidity and mortality. A careful clinical examination was performed on all animals prior to initiation of treatment, and periodically during treatment.

The hamsters were fed ad libitum; weights were monitored once every week and food consumption was measured once a week over a 24 hour period.

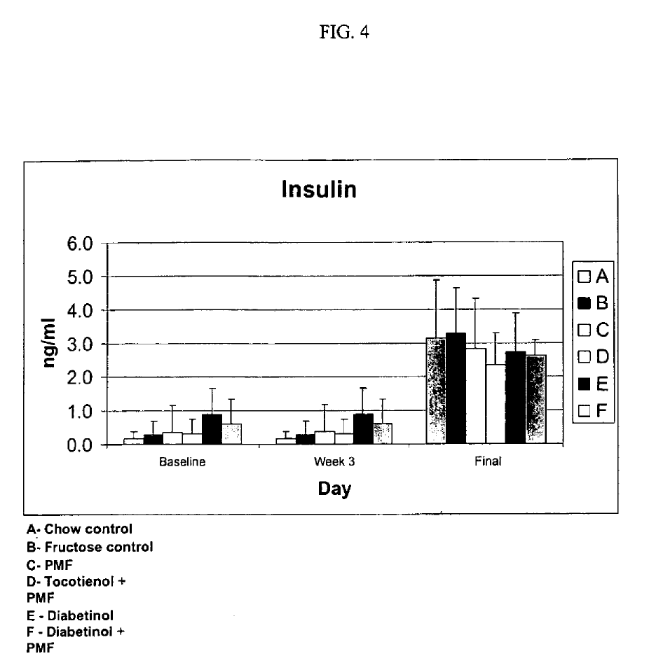

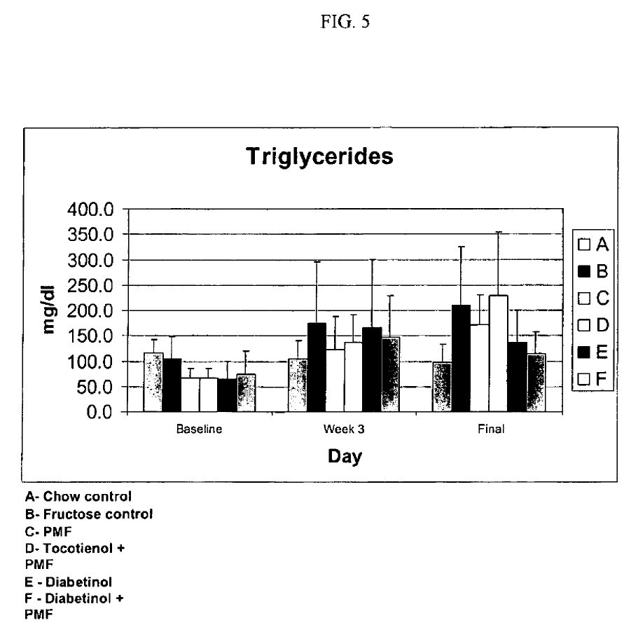

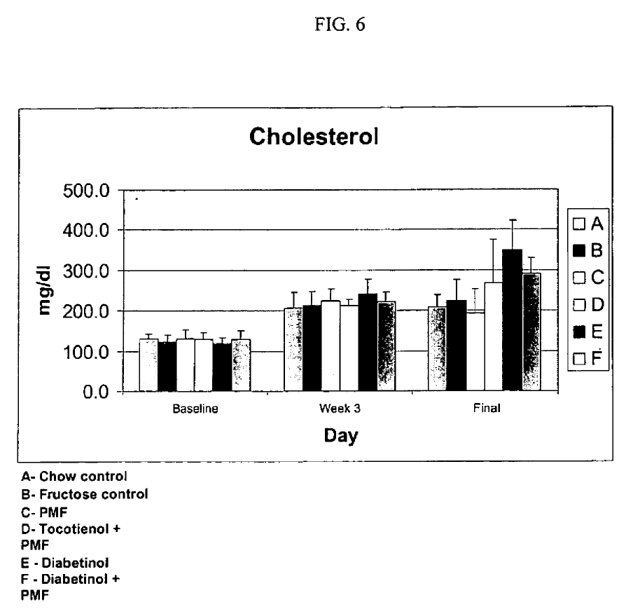

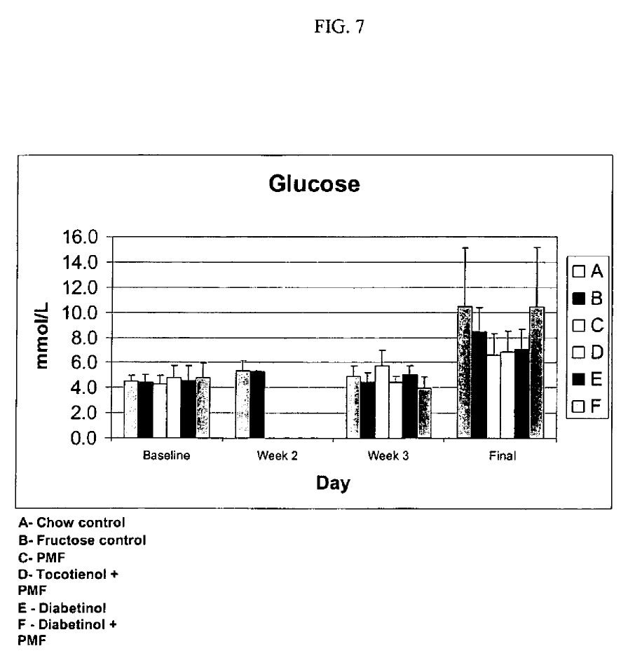

Blood collected during glucose test was tested immediately using glucometer. Triglycerides (Wako, R1-Cat # 998-40391; R2-Cat # 99440491). Total cholesterol levels were determined by enzymatic colorimetric assays using commercially available kit (Wako, Cat # 43917501). Non-Esterified Fatty Acids (NEFA, Wako Cat # 994-75409) and insulin was measured using specific enzyme-linked immunosorbent assays (ELISA; Linco Research, Cat # EZRMI-13K). The data was analyzed using t-test to calculate any significant changes. Test results for insulin, triglycerides, cholesterol and glucose can be seen in the graphs of FIGS. 4, 5, 6 and 7, respectively.

### DISCUSSION AND CONCLUSIONS

Our findings indicate that supplementation with a proprietary formulation might improve glycemic control in individuals with metabolic syndrome. Daily administration of the product for the 4-wk period was associated with more pronounced improvement in insulin vs. glucose responses (the glucose peak tended to be delayed but not reduced whereas the insulin peak tended to be both reduced and delayed).

Positive correlations were found between the degree of improvement in pharmacokinetic parameters (Tₘₐₓ for glucose, AUC₀₋₂ₕ, Cₘₐₓ and Tₘₐₓ for insulin) and subjects' BMI. This suggested that desirable changes in glycemic control induced by treatment were more likely to occur in individuals with higher BMI than in those with lower BMI.

Many other variations of the present invention will be apparent to those skilled in the art and are meant to be within the scope of the claims appended hereto.

1. A pharmaceutical composition comprising an effective amount of a combination of compounds comprising at least one limonoid, at least one flavonoid and at least one tocotrienol, which after about 4 weeks of administration of said composition to humans, said composition provides at least about 10 percent increase in mean time to maximum plasma concentration (T.sub.max) of plasma insulin in said humans after administration of an oral glucose tolerance test as compared to the mean time to maximum plasma concentration (T.sub.max) of plasma insulin after an oral glucose tolerance test prior to said 4 week interval.

2-51. (canceled)
